(19) United States
(12) Patent Application Publication
(10) Pub. No.: US 2004/0247544 A1
(43) Pub. Date: Dec. 9, 2004

(54) Use of dialkyl sulphones in cosmetic nailcare compositions for promoting growth of the nails

(75) Inventors: Elisabeth Picard-Lesboueyries, Velizy (FR); Pascale Bernard, Sucy-En-Brie (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 10/740,769

(22) Filed: Dec. 22, 2003

Related U.S. Application Data
(60) Provisional application No. 60/442,894, filed on Jan. 28, 2003.

(30) Foreign Application Priority Data
Dec. 24, 2002 (FR) 0216651

(51) Int. Cl. A61K 7/04

(52) U.S. Cl. 424/61

(57) ABSTRACT

Use of at least one dialkyl sulphone of formula (I):

R₁—SO₂—R₂ (I)

in which R₁ and R₂, identical or different, represent a straight or branched alkyl group of 1 to 4 carbon atoms, in a cosmetic nailcare composition, applied on the nails, to promote the growth of the nails.

The present invention relates to the use of dialkyl sulphones, and in particular of methylsulphonylmethane, in nailcare compositions, such as nail varnishes, to promote or activate growth of the nails and to increase the rate of growth of the nails.

Nailcare products, such as nail varnishes, may contain various biologically active products, the aim of which is especially to reinforce or harden the nails.

Thus, nailcare products containing silica or formaldehyde as nail hardener are known and marketed.

Other nailcare products contain multivitamin complexes, fluorine, vitamin E or calcium.

No nailcare composition exists that has an actual effect on the growth of the nails and that, possibly, also reinforces the nails.

It has been demonstrated, according to the present invention, that the incorporation of dialkyl sulphones into cosmetic nailcare compositions makes it possible to promote the growth of the nails; in other words, to accelerate the growth of the nails, to increase the rate of growth relative to a normal rate of growth.

In particular, it has been demonstrated, that the incorporation of dialkylsulfones into cosmetic nailcare compositions makes it possible to promote the growth of soft nails.

By soft nails, it is meant a flexible nail, which easily bends, bows, when one tries to bend it.

By hard nail, it is meant a nail exhibiting a resistance when one tries to bend it.

One subject of the invention is thus the use of at least one dialkyl sulphone of formula (I):

R₁—SO₂—R₂ (I)

in which R₁ and R₂, identical or different, represent a straight or branched alkyl group of 1 to 4 carbon atoms, in a cosmetic nailcare composition, applied on the nails, to promote the growth of the nails.

The alkyl group of R₁ and R₂ is chosen, independently, from straight and branched alkyl groups of 1 to 4 carbon atoms, such as methyl, ethyl, isopropyl, n-propyl, n-butyl, isobutyl and tert-butyl groups.

Preferably, R₁ and R₂ are identical.

More preferably, R₁ and R₂ both represent a methyl group and the compound of formula (I) is then dimethyl sulphone or methylsulphonylmethane (MSM).

The compounds of formula (I), and in particular MSM, are known compounds. MSM [67-71-0] has the advantage of being a crystalline white odourless product with a melting point of 109° C. and a boiling point of 238° C.

MSM is present in most green plants, microplankton and algae.

It is a natural compound found in nerve tissues, the skin, the hair and the joints.

MSM has already been used in cosmetic compositions and dietary products.

Thus, document U.S. Pat. No. 4,296,130 describes a cosmetic composition for the skin or the nails, comprising MSM in aqueous or non-aqueous solutions or in emulsion. MSM used in manicure products allows the nails to be strengthened and allows nails that are less brittle to be obtained.

Example 13 of the said document is an aqueous formulation containing 10% MSM and 50% water, which is applied to the nails with cotton wool. This formulation is left to act for 15 minutes. The nails are strong and less brittle.

Example 14 of the said document is a solvent-based (ethyl acetate or amyl acetate) varnish containing 5% by weight of MSM. Application of this varnish makes the nails less brittle.

In Example 15 of the said document, nail-varnish removers are prepared by combining a standard varnish solvent with 5 to 10% of MSM. It is observed that the nails become less brittle.

It is thus known practice from the said document to formulate MSM, without it having necessarily been combined with carbamide and/or DMSO, in a solvent-based varnish or in a water-based varnish, to improve the strength of the nails and to make them less brittle, but the said document does not in any way mention or suggest that MSM has any influence on the growth of the nails.

Document EP-A-0 200 526 relates to the use of MSM in foods, as a source of sulphur, and in the preparation of therapeutic agents, especially antiarthritic, analgesic, antiparasitic and anti-cramp agents administered orally to animals or human beings.

Said document describes, in Example 6, that the oral ingestion of MSM, in the form of an aqueous 5% solution, promotes and stimulates the growth of the nails according to a test performed on dogs.

Although the said document therefore teaches that the administration of MSM orally promotes the growth of the nails, it does not mention or suggest that the topical application of MSM to the nails can lead to such a result. It is known that it is not in any way possible to deduce, on the basis of the efficacy of an orally administered product, that this product will have the same action or the same effect when administered via a different route, for example topically.

Thus, the effect shown of an orally administered product does not in any way anticipate the effect of this same product via the topical route, for example on the nails.

Document U.S. Pat. No. 5,785,959 describes a nail-reinforcing composition comprising:

0.001-20% of a crosslinking thiol agent;

0.1-60% of a “permeation/binding” agent (in particular hydroxy carboxylic acids);

0.001-20% of chelating agent.

Among the crosslinking thiol agents, dimethyl sulphone, or methylsulphonylmethane (MSM) is being cited.

The composition may contain water or oils. It may also be anhydrous, especially in the form of a nail varnish.

The examples teach that the nail-reinforcing composition may be a solvent-based varnish (Example 3) with nitrocellulose and ethyl acetate or butyl acetate (Example 2) or a water-based varnish with an acrylic latex.

In conclusion, the said document thus suggests the use of MSM formulated in a solvent-based varnish or a water-based varnish or with a latex, to reinforce the nails, the reinforcing properties not being otherwise detailed.

Said document does not mention or suggest that MSM has any influence on the growth of the nails promote or activate the growth of the nails is thus neither mentioned nor suggested in the prior art.

In addition, the compounds of formula (I) used in nailcare compositions also make it possible to make the nails harder and less prone to splitting, i.e. the use of the compounds of formula (I) in nailcare compositions also makes it possible to reinforce the nails and to improve the overall appearance of the nails.

Furthermore, the cosmetic properties of the compositions in which the compound of formula (I) is used are not affected and remain excellent. In particular, irrespective of the form of the composition, the properties, such as the gloss, the film tension and the staying power are conserved in is the presence of a compound of formula (I), such as MSM.

The cosmetic nailcare composition comprises the compound(s) of formula (I) in a cosmetically acceptable medium.

The content of compound(s) of formula (I) is generally from 0.05 to 10% by weight and preferably from 0.1% to 4% by weight relative to the total weight of the composition.

The term “cosmetically acceptable medium” means a medium that is compatible with the nails.

The compositions according to the invention may be in any form provided that it is applied to the nails: it may be a simple aqueous solution or a solvent-based solution, of a nailcare cream, of a nail varnish dissolver or of a nail varnish.

Preferably, the cosmetic nailcare composition is a nail varnish.

The cosmetically acceptable medium of the composition may comprise an aqueous medium or an organic solvent medium.

When the composition comprises an organic solvent medium comprising an organic solvent, as organic solvents that may be used in the composition in which the compound of formula (I) is used, mention may be made of:

ketones that are liquid at room temperature, such as methyl ethyl ketone, methyl isobutyl ketone, diisobutyl ketone, isophorone, cyclohexanone and acetone;

alcohols that are liquid at room temperature, such as ethanol, isopropanol, diacetone alcohol, 2-butoxyethanol and cyclohexanol;

glycols that are liquid at room temperature, such as ethylene glycol, propylene glycol, pentylene glycol and glycerol;

propylene glycol ethers that are liquid at room temperature, such as propylene glycol monomethyl ether, propylene glycol monomethyl ether acetate and dipropylene glycol mono-n-butyl ether;

short-chain esters (having from 3 to 8 carbon atoms in total), such as ethyl acetate, methyl acetate, propyl acetate, n-butyl acetate and isopentyl acetate;

alkanes that are liquid at room temperature, such as decane, heptane, dodecane and cyclohexane;

aldehydes that are liquid at room temperature, such as benzaldehyde, acetaldehyde; and

mixtures thereof.

Preferably, the solvent is chosen from short-chain esters (containing from 3 to 8 carbon atoms in total) and alcohols that are liquid at room temperature, and mixtures thereof.

The composition containing an organic solvent medium may further comprise water, especially in a content ranging from 0.1% to 10% by weight relative to the total weight of the composition.

When the composition, such as a nail varnish, comprises an organic solvent medium, the organic solvent may be present in a content ranging from 25% to 94.9% by weight and preferably from 60% to 90% by weight relative to the total weight of the composition.

When the composition, such as a nail varnish, contains an aqueous medium, this medium may consist essentially of water (in particular, the aqueous medium is water) or may comprise a mixture of water and of one or more water-miscible solvent(s), for instance lower monoalcohols containing from 1 to 5 carbon atoms, such as ethanol, glycols containing from 2 to 8 carbon atoms, C₃-C₄ ketones and C₂-C₄ aldehydes.

The water content of the composition may range from 25% to 95% by weight, preferably from 50% to 90% by weight and better still from 60% to 85% by weight relative to the total weight of the composition.

In particular, when the composition is a nail varnish composition, it may contain at least one film-forming polymer in a proportion generally ranging from 1% to 60% by weight and better still from 5% to 45% by weight relative to the total weight of the composition, in an organic solvent medium or an aqueous medium, as defined above.

In the present patent application, the term “film-forming polymer” means a polymer that is capable, by itself or in the presence of a possible plasticizer, of forming an isolable film. The film-forming polymer may be dissolved or dispersed in the form of particles in the cosmetically acceptable medium of the composition.

The film-forming polymer may be chosen from free-radical polymers, polycondensates and polymers of natural origin.

The film-forming polymer may be chosen especially from the group formed by vinyl and acrylic polymers, polyurethanes, polyesters, alkyd resins, epoxyester resins, cellulose-based polymers, such as nitrocellulose, cellulose esters, for instance cellulose acetate, cellulose acetopropionate or cellulose acetobutyrate, resins resulting from the condensation of formaldehyde with an arylsulphonamide, and mixtures thereof.

When the composition, in particular in the form of a nail varnish, comprises an organic solvent medium, the film-forming polymer may be chosen especially from alkyd, acrylic and/or vinyl resins, polyurethanes and polyesters, celluloses and cellulose derivatives, such as nitrocellulose, cellulose esters, such as cellulose acetate, cellulose acetopropionate or cellulose acetobutyrate, resins resulting from the condensation of formaldehyde with an arylsulphonamide, and mixtures thereof.

When the composition, such as a nail varnish, comprises an aqueous medium, then the film-forming polymer is generally present in the form of particles dispersed in the aqueous medium, thus forming a latex or a pseudo-latex.

Among the film-forming polymers that may be used in aqueous medium, mention may be made of polyurethanes, for example anionic polyurethanes, polyester-polyurethanes, polyether-polyurethanes, radical polymers, especially of acrylic, acrylic-styrene and/or vinyl type, polyesters and alkyd resins, alone or as a mixture.

The dispersion may also comprise an associative polymer of polyurethane type or a natural gum, such as xanthan gum.

Aqueous polymer dispersions that may be mentioned include the acrylic polymer dispersions sold under the names Neocryl XK-90®, Neocryl A-1070®, Neocryl A-1090®, Neocryl BT-62®, Neocryl A-1079® and Neocryl A-523® by the company Zeneca, and Dow Latex 432® by the company Dow Chemical. It is also possible to use aqueous polyurethane dispersions, and especially the polyester-polyurethanes sold under the names “Avalure UR-405®”, “Avalure UR-410®”, “Avalure UR-425®” and “Sancure 2060®” by the company Goodrich and the polyether-polyurethanes sold under the names “Sancure 878®” by the company Goodrich and “Neorez R-970®” by the company Avecia.

The film-forming polymer may be present in the composition according to the invention in a solids content, which is effective to obtain a film, generally ranging from 5% to 60% by weight and better still from 10% to 40% by weight, relative to the total weight of the composition.

To improve the film-forming properties of the composition, especially of nail varnish, in which the compound of formula (I) is used, an auxiliary film-forming agent may be provided.

Such an auxiliary film-forming agent may be chosen from any compound known to those skilled in the art as being capable of satisfying the desired function, and may be chosen especially from plasticizers.

In addition, when the base composition according to the invention comprises a film-forming polymer in the form of particles dispersed in an aqueous medium, the auxiliary film-forming agent may also be chosen from coalescers.

The composition may also comprise at least one plasticizer. In particular, mention may be made, alone or as a mixture, of common plasticizers such as:

glycols and derivatives thereof, such as diethylene glycol ethyl ether, diethylene glycol methyl ether, diethylene glycol butyl ether or diethylene glycol hexyl ether, ethylene glycol ethyl ether, ethylene glycol butyl ether and ethylene glycol hexyl ether;

glycols esters;

propylene glycol derivatives and in particular propylene glycol phenyl ether, propylene glycol diacetate, dipropylene glycol butyl ether, tripropylene glycol butyl ether, propylene glycol methyl ether, dipropylene glycol ethyl ether, tripropylene glycol methyl ether, diethylene glycol methyl ether and propylene glycol butyl ether;

acid esters, especially carboxylic acid esters, such as citrates, phthalates, adipates, carbonates, tartrates, phosphates and sebacates;

oxyethylenated derivatives, such as oxyethylenated oils, especially vegetal oils such as castor oil;

mixtures thereof.

The amount of plasticizer may be chosen by a person skilled in the art on the basis of his general knowledge, so as to obtain a composition that has cosmetically acceptable properties. The plasticizer content may range, for example, from 0.1% to 15% by weight and preferably from 0.5% to 10% by weight, relative to the total weight of the composition.

The [symbol] , which dyestuff may be present in a content ranging from 0.01% to 10% by weight relative to the total weight of the composition. The powdery compounds may be chosen from the pigments and/or nacres and/or flakes usually used in cosmetic compositions such as nail varnishes.

The pigments may be white or coloured, and mineral and/or organic. Among the mineral pigments that may be mentioned are titanium dioxide, optionally surface-treated, zirconium oxide or cerium oxide, and also iron oxide, chromium oxide, manganese violet, ultramarine blue, chromium hydrate and ferric blue, and metallic pigments, for instance aluminium or bronze. Among the organic pigments that may be mentioned are carbon black, pigments of D & C type, lakes based on cochineal carmine, on barium, strontium, calcium or aluminium, and guanine.

The nacreous pigments may be chosen from white nacreous pigments such as mica coated with titanium or with bismuth oxychloride, coloured nacreous pigments such as titanium mica with iron oxides, titanium mica with, especially, ferric blue or chromium oxide, titanium mica with an organic pigment of the abovementioned type, and also nacreous pigments based on bismuth oxychloride.

The flakes may be chosen from those made of acrylic resin, polyester, polyethylene terephthalate or of aluminium.

The colorants are, for example, Sudan red, DC Red 17, DC Green 6, β-carotene, soybean oil, Sudan brown, DC Yellow 11, DC Violet 2, DC Orange 5 or quinoline yellow.

The composition may further comprise any other ingredient or additive commonly used in cosmetic compositions and known to those skilled in the art as being able to be incorporated into such a composition, such as a nail varnish.

Such ingredients or additives may be chosen from coalescers, thickeners, preserving agents, fragrances, oils, waxes, surfactants, antioxidants, free-radical scavengers, spreading agents, wetting agents, dispersants, antifoaming agents, neutralizers, stabilizers, active agents chosen from essential oils, UV-screening agents/sunscreens, moisturizers, vitamins, proteins and ceramides; and mixtures thereof.

Needless to say, a person skilled in the art will take care to select this or these optional additional compound(s) and/or the amount thereof, such that the advantageous properties associated with the use in the composition according to the invention of a compound of formula (I) are not, or are not substantially, adversely affected by the envisaged addition.

The composition in which the compound of formula (I), such as MSM, is used is in one of the forms already described above. The composition may be applied to the nails via any suitable means, such as an applicator (“pinceau”), a brush, a pad, a wipe (towelette), etc.

The invention will now be described with reference to the examples that follow, which are given as non-limiting illustrations.

### DETAILED DESCRIPTION OF PARTICULAR EMBODIMENTS

Examples 1 and 2 below illustrate the preparation of cosmetic nailcare compositions in which is incorporated a compound of formula (I), namely MSM.

### Example 1

Water-based Varnish Containing MSM.

A water-based varnish formulation is prepared, containing 89.47% of a polyurethane latex (Cydrothane HP 6000 from the company Cytec), to which is added a coalescer (dipropylene glycol monobutyl ether sold by the company Dow under the name “Dowanol DPnB”) (1.1%), followed by preserving agents (0.54% of sodium methyl paraben and 0.4% of diazolidinylurea), 0.05% of surfactant to facilitate the peeling and spreading of the film (polyfluorohexyl betaine such as Amphoram® from the company CECA®), ethanol (4.36%), 0.09% of associative polyurethane (Serad® FX 1100 from the company Servo Delden) and 2% of MSM ground mechanically beforehand with a mortar. To speed up dissolving the MSM in the above mixture, the whole mixture may be heated in a water bath (at about 50° C.).

### Example 2

Solvent-based Varnish Containing MSM

0.5% of MSM are added to nitrocellulose (10 g), premixed with ethyl acetate; next, after total dissolution, a resin of alkyd type, a plasticizer (acetyl tributyl citrate sold under the reference Citroflex® by the company Pfizer) (content of resin and plasticizer equal to 15 g) are added and the mixture is finally completed with the two solvents (ethyl acetate and butyl acetate, qs 100 g).

Needless to say, the two formulations of Examples 1 and 2 may contain pigments, colorants, nacres and other additives.

1-26. (Cancelled)

27. A method to promote growth of the nails, comprising applying to a nail a nailcare composition comprising at least one dialkyl sulphone of formula (I)
R₁—SO₂—R₂ (I)
in which R₁ and R₂, which are identical or different, represent a straight or branched alkyl group of 1 to 4 carbon atoms.

28. The method according to claim 27, in which the alkyl group of R₁ and R₂ is chosen, independently, from methyl, ethyl, isopropyl, n-propyl, n-butyl, isobutyl and tert-butyl groups.

29. The method according to claim 27, in which R₁ and R₂ are identical.

30. The method according to claim 29, in which R₁ and R₂ both represent a methyl group.

31. The method according to claim 27, wherein said application makes the nail harder.

32. The method according to claim 27, in which the content of compound of formula (I) in said composition is from 0.05% to 10% by weight relative to the total weight of the composition.

33. The method according to claim 27, in which the composition further comprises a cosmetically acceptable medium.

34. The method according to claim 1, in which the composition further comprises an organic solvent medium comprising an organic solvent.

35. The method according to claim 34, in which the organic solvent is selected from the group consisting of:
ketones that are liquid at room temperature;
alcohols that are liquid at room temperature;
glycols that are liquid at room temperature;
propylene glycol ethers that are liquid at room temperature;
short-chain esters having from 3 to a carbon atoms in total;
alkanes that are liquid at room temperature;
aldehydes that are liquid at room temperature;
mixtures thereof.

36. The method according to claim 34, in which the organic solvent is selected from the group consisting of from short-chain esters, alcohols that are liquid at room temperature, and mixtures thereof.

37. The method according to claim 34, in which the organic solvent is present in a content ranging from 25% to 94.9% by weight relative to the total weight of the composition.

38. The method according to claim 27, in which the composition further comprises an aqueous medium.

39. The method according to claim 38, in which the aqueous medium consists essentially of water or of a mixture of water and one or more water-miscible solvents.

40. The method according to claim 38, in which the water content of the composition is from 25% to 95% by weight.

41. The method according to claim 27, in which the composition further comprises at least one film-forming polymer in a proportion of from 1% to 60% by weight relative to the total weight of the composition.

42. The method according to claim 41, in which the film-forming polymer is selected from the group consisting of free-radical polymers, polycondensates and polymers of natural origin.

43. The method according to claim 27, in which the composition further comprises an organic solvent medium and a film-forming polymer selected from the group consisting of alkyd, acrylic and/or vinyl resins, polyurethanes and polyesters, and celluloses and cellulose derivatives.

44. The method according to claim 41, in which the film-forming polymer is selected from the group consisting of polyurethanes, radical polymers, polyesters, alkyd resins, and mixtures thereof.

45. The method according to claim 41, in which the composition further comprises an auxiliary film-forming agent.

46. The method according to claim 27, in which the composition further comprises at least one plasticizer.

47. The method according to claim 27, in which the composition further comprises a dyestuff.

48. The method according to claim 47, in which the dyestuff is a powdery compound and/or a dye that is soluble in the composition.

49. The method according to claim 27, in which the composition further comprises an additive selected from the group consisting of coaleacers, thickeners, preserving agents, fragrances, oils, waxes, surfactants, antioxidants, free-radical scavengers, spreading agents, wetting agents, dispersants, amtifoaming agents, neutralizers stabilizers, active agents chosen from essential oils, tiv-screening agents/sunscreens, moisturizers, vitamins, proteins, ceramides; and mixtures thereof.

50. The method according to claim 27, in which the composition is in the form of an aqueous solution or a solution in a solvent, of a nailcare cream, of a nail varnish or of a nail varnish dissolver.

51. The method according to claim 27, in which the composition is a nail varnish.
